(19) United States
(12) Patent Application Publication
(10) Pub. No.: US 2011/0305738 A1
(43) Pub. Date: Dec. 15, 2011

(54) Oxygenating Oral Compositions

(76) Inventor: Daniel A. Ladizinsky, Lake Oswego, OR (US)

(21) Appl. No.: 13/155,432

(22) Filed: Jun. 8, 2011

Related U.S. Application Data
(60) Provisional application No. 61/352,986, filed on Jun. 9, 2010.

(51) Int. Cl. A61K 8/11; A61K 8/02; A61Q 11/00; A61K 8/66; A61K 8/22; B29C 47/06; A61K 9/28

(52) U.S. Cl. 424/401; 156/244.11; 424/48; 424/53; 427/2.14

(57) ABSTRACT

Oral compositions, which are preferably chewable compositions, containing a peroxide source and a peroxide decomposition catalyst in a contactless manner liberate molecular oxygen in the oral cavity upon use by catalyzed decomposition of the peroxide source. The oral composition promotes oral health by providing a more aerobic environment for oral flora.

### CROSS-REFERENCE TO RELATED APPLICATION

This application claims priority to provisional application 61/352,986 filed Jun. 9, 2010 which is herein incorporated by reference.

### BACKGROUND OF THE INVENTION

1. Field of the Invention

The invention pertains to oxygenating oral compositions, preferably a gum, gel or lozenge, which release oxygen in the oral cavity, preferably, in the case of a gum during mastication, thus promoting the health of the oral cavity.

2. Background Art

Sugar, saliva, and anaerobic bacteria lead to a formidable combination that may lead to tooth decay. After eating sugar, particularly sucrose, and even within minutes of brushing the teeth, sticky glycoproteins (combinations of carbohydrates and protein molecules) adhere to the teeth to start the formation of plaque biofilm. At the same time millions of bacteria known as Streptococcus mutans also adhere to the glycoprotein. Although many other oral bacteria also adhere, only the S. mutans is able to cause cavities. In the next stage, the bacteria metabolize fructose in a glycolysis process. The end product of glycolysis under anaerobic conditions is lactic acid. The lactic acid creates extra acidity to decrease the pH to the extent of dissolving the calcium phosphate in the tooth enamel leading to the start of a cavity.

In addition to tooth decay, anaerobes that are part of the indigenous flora of the oral cavity can be recovered from various infections adjacent to that area, such as infectious cervical lymphadenitis, subcutaneous abscesses, and infected burns in proximity to the oral cavity; infected human and animal bites; paronychia; tonsillar and retropharyngeal abscesses; chronic sinus infection; chronic otitis media; periodontal abscess; infectious thyroiditis; aspiration pneumonia; empyema; and bacteremia associated with one of the above infections. The predominant anaerobes recovered from these infections are species of anaerobic gram-negative bacilli (including pigmented Prevotella and Porphyromonas, Prevotella oxalis and other Prevotella species, and Fusobacterium) and gram-positive anaerobic cocci (Peptostreptococcus species), which are all part of the normal flora of the mucosal surfaces of the oral, pharyngeal, and sinus cavities.

There are several antibiotic based strategies aimed at reducing anaerobic bacterial counts in addition to normal dental hygiene. It is important to note that all antibiotic based strategies, if used chronically, will lead to resistant flora. Signoretto and Ahn, as disclosed in C. Signoretto et al., “Microbiological evaluation of the effects of hyperbaric oxygen on periodontal disease”, NEW MICROBIOLOGICA 30(4): 431-7 (2007) and S. Ahn, et al., “Effect of oxygen on virulence traits of Streptococcus Mutans”, J. BACTERIOL, Dec. 2007, 189(23), 8519-27, have reduced periodontal disease by using perfluourocarbons and even hyperbaric oxygen, but the need for a simpler method is present. Others have demonstrated some efficacy of a chewing gum containing urea hydrogen peroxide. H. Etemadzadeh, “Plaque growth inhibiting effect of chewing gum containing urea hydrogen peroxide,” J. CLIN. PERIODONTAL, 18(5), 337-40 (May 1991).

The use of urea peroxide (“carbamic peroxide”) in chewing gum is also disclosed in Goulet U.S. Pat. No. 5,500,207 along with other peroxides, for the purpose of teeth whitening. Theisen U.S. Pat. No. 5,972,374 discloses a cylindrical chewing gum with separable portions, the cylinder having a central area containing a teeth whitening agent which may contain carbamic peroxide. Mishewitz U.S. Pat. No. 5,693,334 discloses slow release gum formulations containing encapsulated sodium bicarbonate and a peroxygen compound such as carbamic peroxide. Montgomery U.S. Pat. No. 5,908,614 discloses an oral care composition which contained a hydrogen peroxide precursor and an activator which stimulates production of peroxidate enzyme in the oral cavity to generate hydrogen peroxide from the peroxide precursor.

With the exception of the methods disclosed by Signoretto and Ahn, which elevate the oxygen tension in the oral cavity, by means clearly not useable in the absence of a clinical setting, the other references use peroxides for whitening or as biocides.

A chewing gum or equivalent device that produces oxygen in the oral cavity will be beneficial to dental and oral cavity health by changing the anaerobic microenvironment of the periodontal region. This will inhibit the anaerobic bacteria and therefore the deleterious effects of their anaerobic metabolic byproducts such as lactic acid. It has been observed that the ability of S. mutans to form biofilms is severely impaired by oxygen, as disclosed by Ahn. Since S mutans is a facultative anaerobe, it likely will not be eliminated by elevated oxygen levels, but if it switches to aerobic metabolism rather than anaerobic glycolysis, the detrimental lactic acid byproduct will not be present to the same degree and there will be less effects on the dental enamel. Also reduction in anaerobic bacteria may reduce infectious or inflammatory conditions elsewhere in the body.

### SUMMARY OF THE INVENTION

The present invention is directed to an oral composition, preferably a chewable composition, which contains a peroxide and a catalyst for its decomposition to generate molecular oxygen. The oxygen thus generated creates a more aerobic environment which lessens the ability of S. mutans to use anaerobic glycolysis to produce lactic acid. The catalyst and peroxide are kept separate in the composition prior to use.

### DETAILED DESCRIPTION OF THE PREFERRED EMBODIMENTS

The compositions of the present invention may be in the form of a masticatable gum, wax, gel, or the like, all termed collectively “chewable composition” hereafter, unless noted otherwise. The preferred compositions are chewing gums, the formulations of which are well known, as illustrated by U.S. Pat. Nos. 6,696,043; 5,405,623; 5,1992,562; 5,085,872; 5,145,696; and 4,986,991, all incorporated herein by reference. The oral composition may also be in the form of a lozenge which may slowly dissolve in the oral cavity, with or without chewing. Chewable gums are preferred.

Traditionally the chewing gum base may be a natural gum based on rubber latex, or a variety of synthetic polymers such as homo- and co-polymers based on polyvinyl acetate, with comonomers such as ethylene, vinyl propionate, and vinyl laurate. Chewing gum bases are typically natural products, usually extracts from certain trees, plants, and microbes. They are usually divided into three categories: soft, medium, and hard. The soft and medium varieties are preferred for use in the chewing gum formulations herein. The chewing gum bases commonly used in conventional chewing gum are suitable as ingredients of the formulations of the preferred embodiment. In one embodiment, the chewing gum base is added at between 50 and 90%, including 55%, 60%, 65%, 70%, 80%, and 85% by weight. In a further embodiment, the chewing gum base is added at between 65% and 85%. In a further embodiment, the chewing gum base is added at between 70% and 80%. It is to be understood that substantial quantities of chewing gum additives are used, the concentration may vary.

Conventional excipients such as sweeteners, flavors, inorganic fillers, and such may be added. In the present chewable compositions, although natural sweeteners such as sucrose, maltose, glucose, fructose, etc., may be used, these are often not preferred. Rather, an artificial sweetener is often preferred, as these do not supply a further energy source for S. mutans.

The peroxide may be any peroxide which is safe for human consumption and which will liberate oxygen in the presence of a suitable catalyst. Solid peroxides are preferred, of which there are many, including percarbonates and perborates. Suitable solid peroxides are sodium percarbonate, carbamic peroxide and calcium peroxide. Liquid peroxides may also be used, for example hydrogen peroxide. When liquid peroxides are used, it is preferred that they are present in encapsulated form, preferably with a water soluble or biodegradable polymer such as polyvinylalcohol, polyvinylpyrollidone, or a natural coating such as crosslinked or non-crosslinked gelatin. Methods of encapsulating liquids with such coatings are well known in the art, and are disclosed, for example, in U.S. Pat. Nos. 5,908,614 and 5,693,334, which are incorporated herein by reference. “Liquid peroxides” as used herein also include solutions of solid peroxides.

Preferred hydrogen peroxide sources are sodium percarbonate and carbamide peroxide due to their solubility characteristics and relatively benign toxicity in limited concentrations. The most preferred non-enzymatic hydrogen peroxide precursor is sodium percarbonate. Calcium peroxide is also a preferred peroxide source.

Sodium percarbonate is a relatively stable complex containing 2 moles of sodium carbonate complexed with 3 moles of hydrogen peroxide (27% hydrogen peroxide by weight). It is highly water soluble (120 grams per liter at 20.degree. C.) and produces a pH upon dissolution of between 10 and 11 (for a 1% solution). Thus, although sodium percarbonate possesses the desirable hydrogen peroxide-releasing properties, alone they are of little utility for the activation of a peroxidase enzyme due to their high in-solution pH properties. Accordingly, a pH adjusting agent is preferably utilized to normalize the pH to a range of 4.0-7.9.

Carbamide peroxide is a 1 to 1 molar complex between urea and hydrogen peroxide (35% hydrogen peroxide by weight) with a molecular weight of 94.07. It is usually manufactured in the form of crystals which are highly soluble in water (800 grams per liter of water at 20° C. to yield a saturated solution of 44.4% carbamide peroxide, equivalent to a hydrogen peroxide concentration of 15.5%). However, when carbamide peroxide is solubilized in water, a pH of approximately 3.40 (for a saturated solution) to approximately 4.05 (for a 1% solution) is obtained. This pH is slightly below the desirable range, for activating a peroxidase enzyme in the aqueous contact solution absent a pH adjusting agent, so addition of a pH adjusting agent is preferred.

The chewable compositions should thus not be too basic or too acidic, to avoid irritation of the lining of the oral cavity and the tongue. Furthermore, when biological enzymes are used as peroxide decomposition catalysts, each enzyme has a range of pH in which it is most effective. Thus, the chewable composition may contain basic substances such as sodium or potassium carbonates or bicarbonates, calcium carbonate, calcium or magnesium hydroxide, alkali metal phosphates and hydrogen phosphates, alkali metal acetates and propionates, etc., to lower acidity, and acidic substances such as alkali metal dihydrogen phosphates, ammonium halides and sulfates, carboxylic acids such as acetic acid, propionic acid, and mineral acids in minor quantities, to lower basicity. Buffer preparations are also useful in such compositions.

The peroxides are preferably at concentrations corresponding to 1-20% by weight hydrogen peroxide. Preferably calcium peroxide is used at concentrations corresponding to 3 to 7% calcium peroxide, including 3.5%, 4%, 4.5%, 5%, 5.5%, 6%, and 6.5%. In other embodiments, the calcium peroxide is used at 3.4 to 4.6% calcium peroxide. The peroxides may also be mixed and include 2, 3, 4, or more different peroxides in any ratio. In one embodiment a mixture of calcium peroxide and zinc peroxide is used and the zinc peroxide is at a concentration of about 0.3 to 0.7% by weight.”

The catalyst may be a naturally occurring or synthetic enzyme such as catalase, superoxide dismutase, salivary peroxidase, myeloperoxidase, glutathione peroxidase or other, or an inorganic catalyst such as manganese dioxide, or an alkali or alkaline earth permanganate or other. The preferred catalyst is catalase due to its low toxicity and high stability.

Optional but desirable additives to the formulation of the preferred embodiments which may have beneficial effects on the rate of decomposition of hydrogen peroxide by the catalyst during the mastication process include, but are not limited to, hydroxides, oxides, and salts of alkaline earth metals, particularly carbonates; hydroxides and carbonates of sodium, calcium and potassium; silicas; and calcium silicate. In one embodiment, the calcium carbonate is added at between 0.5% and 6% by weight, including 1.5%, 2%, 2.5%, 3.5%, 4%, 4.5%, and 5%. In a further embodiment, the calcium carbonate is added at between 1% and 3% by weight.

Hydrophilic additives may be added to the chewing gum base. Examples of hydrophilic additives which may be used includes glycerin, propylene glycol, and polyglycols.

Flavoring and coloring agents may be added to enhance the acceptance or appeal of either or both parts, or as indicators of the reactivity of peroxide and the progress of radical oxygen generation. The most desirable flavors may include, among others, food grade orange, lemon, peppermint, spearmint, mint, bubble gum, cherry, watermelon, strawberry and apple varieties. As coloring agents FD&C or FD&C water soluble dyes may be used; FD&C Blue #1 and FD&C Blue #2 are preferred. In one embodiment, the flavor additive is peppermint oil and it is added at between 0.05 and 0.5%, including 0.075%, 0.15%, 0.2%, 0.3%, 0.4%, and 0.45%. In a further embodiment, the peppermint oil or other flavor additive is added at between 0.1 and 0.4%.

Although it is to be understood that the strengths of flavoring differ considerably a much lower or higher concentration may be needed.

Sweeteners may be added to the chewing gum including sucrose, saccharine, aspartame, fructose, xylitol, sorbitol and mixtures thereof. Preferably, the sweetener is xylitol or fructose. In one embodiment, the xylitol, fructose or other sweetener is added at between 1 and 10%, including 2%, 3%, 4%, 5%, 6%, 7%, 8%, and 9%. In a further embodiment, the xylitol, fructose, sorbitol or other sweetener is added at between 4 and 8%. In a further embodiment, the sweetener is added at between 5 and 8%. Although it is to be understood that the strength of sweeteners known to one of skill in the art varies considerably and a much lower concentration may be needed, depending on the sweetener used. Xylitol is a preferred sweetener as it has been shown to have benefit in reduction of oral biofilm plaque.

The peroxide and catalyst must be kept separated in the oral compositions, as otherwise premature oxygen generation will occur, resulting in a short shelf life. Any suitable separating method may be used. By the term “substantial contact” as used herein is meant a degree of contact which provides for a storage stable product. This product preferably allows for decomposition of no more than 10% of the peroxide source when stored at ambient temperature (20-25° C.) and 50% relative humidity for one month. Preferably, no more than 10% will decompose over a 6 month period or longer. Most preferably, the composition is stable to peroxide decomposition by the catalyst.

Methods of encapsulation are well known. The encapsulating material forms a coating or wall around the component to be encapsulated. The thickness of the wall may be adjusted to take into account the processing methods used to prepare the oral compositions. For example, in a highly viscous gum base, the walls of the encapsulant may constitute from 20 to 40 weight percent by weight of the encapsulated material, by way of example and not by way of limitation, whereas in less viscous gums, in gels, or in lozenges where less shear and less pressure during processing is to be expected, much thinner walls may be appropriate, even approaching a mono- or bi-molecular layer. The encapsulant is selected such that it is not soluble by the oral composition ingredients. In the case of dissolvable tablets or lozenges, the encapsulant should be water soluble. Methods of encapsulation are, as indicated, well known. Suitable methods may be found, for example, in S. J. Risch, ENCAPSULATION AND CONTROLLED RELEASE OF FOOD INGREDIENTS, ACS Symposium Series, Vol. 590© 1995, American Chemical Society, ISBN 13; 9780841231641, and numerous other references.

In one embodiment, the chewable composition may be gum in the form of “sticks”, essentially flat strips. This form of chewing gum is widely used, and is prepared by extrusion of the gum material through a die. Prior to extrusion, sweeteners, flavorants, fillers, etc., are added to the gum base separately, i.e. in a dough mixer, Banbury mixer, or the like, or may be added to a single or twin screw extruder and mixed in the extruder, prior to exiting the die. In a preferred embodiment, the gum is coextruded as contacting strips, a first strip containing the catalyst, and a second strip containing the peroxide. Prior to contacting the two strips to form an integral “stick” product, a barrier layer of a water soluble polymer may be applied between the strips if necessary. In a further embodiment, a third strip, containing no catalyst and no peroxide source is coextruded between first and second strips. The strips thus extruded may be self-adherent, or may require application of modest pressure, i.e. be passing through one or more roll nips, to produce a stick which will not easily separate into its component strips.

In a further embodiment, a single strip is used, which may also take the form of a lozenge or other shape, for example by a pelletization or injection molding process. In this embodiment, the separation of catalyst and peroxide may be accomplished by encapsulating one or the other of these components, or both components, prior to admixture to the gum base. In addition to encapsulation, one of the catalyst or peroxide may also be dispersed, generally uniformly, into a polymer which will liberate the component during mastication, in an aqueous environment. For example, a water soluble polymer such as a polyacrylic acid or salt thereof can be used for this purpose. A gum material of a “harder” nature than the gum base may also be used. For example, a component may be blended, extruded, and pelletized in a gum base with a higher Tg than the bulk of the gum base, such that upon blending with the conventional gum base, the pellets or granules substantially survive the gum extrusion process.

When embodied in a candy or “soft candy” preparation, pellets, each containing but one of the catalyst and peroxide can be positioned adjacent each other in the form of the large “pellets” or intermixed with each other in granules, and encased with a hard shell.

The above modifications are also useful especially in gels, wherein the gel may contain one component of catalyst or peroxide, and the remaining component is contained in encapsulated form or dispersed in polymer pellets. Suitable gels are well known, and include those preparable from natural sources such as gelatins, starches, vegetable gums such as gum tragacanth or gum agar, etc. Synthetic gels such as those based on chemically modified celluloses, etc., may also be useful. The catalyst or peroxide, if the latter is liquid, may also be supplied as a complex with cyclodextrin. The chewable composition may conveniently be in cylindrical form as disclosed in U.S. Pat. No. 5,972,374.

When the oral composition is in the form of a lozenge, the same considerations apply as applied to the chewable compositions. The lozenges may be coated, i.e. with a soluble “protective” coating such as a polyvinyl alcohol, or with a candy coating. The lozenges contain at least one of the peroxide source and peroxide catalyst in an encapsulated form, or in another form which keeps the peroxide and catalyst separate until use. If encapsulated, the walls of the encapsulant are water soluble, so that they dissolve in the oral cavity, releasing their active ingredients. Preferred wall materials of the encapsulated peroxide and/or catalyst are coatings of sugars, starches, gelatins, or water soluble synthetic polymers such as polyvinylalcohols, polyvinylpyrollidones, polyacrylic acids, and the like. Of course, if the lozenge is also masticated, mastication will enhance freeing of the encapsulated peroxide or catalyst. However, since lozenges are ordinarily placed in the mouth and manipulated by the tongue, etc., their principle mode of use involves dissolution rather than mastication. As the encapsulated component(s) dissolve, the catalyst and peroxide come into contact, generating molecular oxygen. The lozenge may be in the form of a hard candy, for example by incorporating a relatively large amount of a sugar such as xylitol, or may be in the form of a tablet composed of softer ingredients. In tablets, a larger amount of filler may advantageously be present. The filler may also serve as pH-adjusting material when appropriate. Calcium carbonate is such a filler, for example, which is mildly basic, while silicic acid, preferably in the form of precipitated or colloidal silica, is mildly acidic. A binder such as sugar, starch, gelatin, or adhesive polymer may be present.

The subject invention further relates to processes for preparing the oral compositions, and to their use in mammalian species for elevating the oxygen content of the oral cavity. The oral compositions are useful for veterinary purposes, i.e. for dogs, cats, etc., as well as in humans.

The oral compositions of the subject invention have the advantage that the oxygen liberated may change the metabolism of S. mutans to lessen production of lactic acid, without giving rise to drug resistant mutations as with the use of antibiotics. The subject invention has the further advantage that peroxide is present to act as an biocide, prior to its decomposition into oxygen. Thus, the use of the peroxide source with catalyst provides a synergistic effect of reducing oral flora while also creating an aerobic environment.

Alternatively, the substrate and catalyst could be embodied in a lozenge or troche or a chewable/swallowable hard or soft candy. For veterinary application, the components could be embodied within a rawhide or other long lasting chew vehicle.

These embodiments rely on the agitation within the mouth to mix the components. The oxygen produced will be dissolved in water and as such will travel in the oral cavity fluid into interstices within the periodontal tissues. The substrate catalyst combination will become more in contact with progressively more chewing or manipulation within the mouth, until eventually all substrate will be converted to oxygenated water. Normal salivary pO₂ mirrors venous blood pO₂ and ranges from 33-44 mmHg. 100% oxygen saturation in aqueous solution at body temperature occurs at about 120 mmHg. Once that level is exceeded, oxygen bubbles will form and leave solution. Embodiments can be designed that will release oxygen slowly or quickly, depending on the desired effect.

Rapid production for quick release may produce bubbling or a perception of a tingling effervescence and slow production for sustained release may have no tactile perception, but rather a prolonged effect on the perioral tissues. Embodiments may have an initial effervescence, then a later sustained release by keeping some substrate or catalyst more concealed by a method such as microencapsulation, which may require more mastication to release the contents.

Regular use of these products will provide a pleasurable gum chewing experience while providing reduction in the incidence of tooth decay and anaerobe based perioral diseases.

While embodiments of the invention have been illustrated and described, it is not intended that these embodiments illustrate and describe all possible forms of the invention. Rather, the words used in the specification are words of description rather than limitation, and it is understood that various changes may be made without departing from the spirit and scope of the invention.

What is claimed is:

1. An oral composition containing a peroxide source and a peroxide decomposition catalyst which catalyzes the decomposition of the peroxide source to produce molecular oxygen, the peroxide source and peroxide decomposition catalyst present in the oral composition in a non-contacting manner such that no substantial contact of the peroxide source and peroxide decomposition catalyst occurs prior to use of the chewable composition.

2. The composition of claim 1, which is a chewable composition.

3. The chewable composition of claim 2, which is a gum, wherein at least one of the peroxide source and the peroxide decomposition catalyst are encapsulated to prevent substantial contact prior to chewing.

4. The chewable composition of claim 2, which is a multi-part gum, a first part containing the peroxide source and a second part containing the peroxide decomposition catalyst.

5. The chewable composition of claim 4, wherein said first and second parts are extruded through a die and assembled into a unitary structure side by side.

6. The chewable composition of claim 5, wherein between said first part and said second part is a third part containing no peroxide source and no peroxide decomposition catalyst.

7. The chewable composition of claim 3 wherein the gum is in the form of a stick or in the form of a coated solid.

8. The oral composition of claim 1, wherein the peroxide source is selected from the group consisting of alkali metal percarbonates, alkali metal perborates, calcium peroxide, hydrogen peroxide, carbamic peroxide, and mixtures thereof.

9. The oral composition of claim 1, wherein the peroxide decomposition catalyst comprises an enzyme which catalyzes peroxide decomposition.

10. The chewable composition of claim 3, wherein the peroxide decomposition catalyst comprises an enzyme which catalyzes peroxide decomposition.

11. The oral composition of claim 9, wherein the peroxide decomposition catalyst comprises catalase.

12. The oral composition of claim 1, further containing a pH-adjusting agent to adjust the pH of the chewable composition to between a pH of 4.0 and 7.9.

13. The oral composition of claim 1, which is in the form of a lozenge, wherein at least one of the peroxide source and the peroxide decomposition catalyst are encapsulated by a water-soluble encapsulant.

14. A method of increasing the oxygen content of an oral cavity, comprising masticating a chewable composition of claim 1, wherein mastication causes said peroxide source and said peroxide decomposition to come into contact, whereby molecular oxygen is liberated by decomposition of said peroxide source.

15. A method of increasing the oxygen content of an oral cavity, comprising dissolving in the oral cavity a lozenge of claim 1, wherein dissolving causes said peroxide source and said peroxide decomposition to come into contact, whereby molecular oxygen is liberated by decomposition of said peroxide source.

16. A process for the preparation of a chewable composition of claim 2, comprising extruding a first composition comprising a gum base and a peroxide source into a first extrudate, extruding a second composition comprising a gum base and a peroxide decomposition catalyst into a second extrudate, and contacting said first extrudate and said second extrudate without substantial mixing.

17. A process for the preparation of an oral composition of claim 1 which is a gel, comprising forming a first gel comprising a gelling material and a peroxide source, forming a second gel containing a gelling material and a peroxide decomposition catalyst, and forming a chewable product containing both said first gel and said second gel.

18. A process for the preparation of an oral composition of claim 1 which is a lozenge, comprising encapsulating at least one of a peroxide source and a peroxide decomposition catalyst with a water soluble encapsulant, and forming a lozenge containing the peroxide source and peroxide decomposition encapsulated with a water soluble encapsulant.
